(19) United States
(12) Patent Application Publication
(10) Pub. No.: US 2020/0138626 A1
(43) Pub. Date: May 7, 2020

(54) PERCUTANEOUS FRONTAL ACCESS FOR TREATING OCCLUSION OF THE OPHTHALMIC ARTERY

(71) Applicant: J.D. Franco & Co., LLC, Plano, TX (US)

(72) Inventors: Jeff Franco, Plano, TX (US); Michael Calhoun, Lighthouse Point, FL (US)

(73) Assignee: J.D. Franco & Co., LLC, Plano, TX (US)

(21) Appl. No.: 16/670,456

(22) Filed: Oct. 31, 2019

Related U.S. Application Data
(60) Provisional application No. 62/754,099, filed on Nov. 1, 2018.

(51) Int. Cl. A61F 9/007

(52) U.S. Cl. CPC A61F 9/007; A61M 2210/0612

(57) ABSTRACT

A method is provided and may include accessing a terminal branch of an ophthalmic artery through a skin of a head of a subject via a first device, positioning at least one of the first device or a second device within the ophthalmic artery of the subject, and performing an atherectomy of the ophthalmic artery or a junction between the ophthalmic artery and an internal carotid artery of the subject, using the at least one of the first device or the second device, so as to treat a blockage, a stenosis, a lesion, plaque, or other physiology in the ophthalmic artery or the junction between the ophthalmic artery and the internal carotid artery of the subject.

### CROSS-REFERENCE TO RELATED APPLICATIONS

This application claims the benefit of priority of U.S. Provisional Patent Application No. 62/754,099, filed on Nov. 1, 2018, which is incorporated by reference herein in its entirety.

### FIELD

The present disclosure relates to devices and methods of performing ophthalmic artery atherectomy, and, in particular, to devices and methods for accessing the ophthalmic artery via the supratrochlear artery, the dorsal nasal artery, or the supraorbital artery.

### BACKGROUND

Ischemic damage to the eye and/or brain is a significant risk associated with directional coronary atherectomy to remove plaque from narrowed blood vessels in the heart. However, when confronted with plaque of the ophthalmic artery (OA) and other vasculature of the eye, researchers have attempted instead to deliver radiation therapy to the subretinal neovascularization that is associated with macular degeneration, rather than attempt atherectomy of such vessels. A problem with such an attempt is the lack of devices capable of accessing such narrow peripheral vasculature. An additional problem is the large ischemic risk associated with treating vessels that are so near the brain.

Diseases of the eye, specifically, wet age-related macular degeneration (WAMD), glaucoma, and diabetic retinopathy, affect a large percentage of the population. Currently approved treatments include surgically implanting a miniature lens (VisionCare), injecting the anti-cancer drug Avastin® into the eye on a monthly basis, injecting a therapeutic antibody into the eye (e.g., Macugen®, pegaptanib), and photo or laser treatment to destroy “abnormal” blood vessels. However, current therapies are deficient in one or more aspects, necessitating improved approaches. The present disclosure addresses some or all of the problems found in current therapies.

### SUMMARY

The present disclosure relates to, for example, devices and methods for percutaneous frontal access via the supratrochlear or the supraorbital artery for atherectomy treatment of the ophthalmic artery and vascular structures in the rear of the eye, including treatment for the symptoms related to WAMD by removal of stenosis of the OA, thereby restoring normal, or near normal, blood flow to the rear of the eye.

In one embodiment, there is provided a method which may include: accessing a terminal branch of an ophthalmic artery through a skin of a head of a subject via a first device; positioning at least one of the first device or a second device within the ophthalmic artery of the subject; and performing an atherectomy of the ophthalmic artery or a junction between the ophthalmic artery and an internal carotid artery of the subject, using the at least one of the first device or the second device, so as to treat a blockage, a stenosis, a lesion, plaque, or other physiology in the ophthalmic artery or the junction between the ophthalmic artery and the internal carotid artery of the subject.

In some embodiments, the step of performing the atherectomy includes increasing a size of a lumen of the ophthalmic artery or the junction between the ophthalmic artery and the internal carotid artery of the subject.

In some embodiments, the step of accessing the terminal branch of the ophthalmic artery through the skin of the head of the subject includes accessing one of a supraorbital artery, a dorsal nasal artery, or a supratrochlear artery of the subject via the first device.

In some embodiments, the method also may include measuring a blood flow rate in the ophthalmic artery.

In some embodiments, the method also may include inhibiting antegrade passage of debris via a distal protection element.

In some embodiments, the method also may include performing an angioplasty of the ophthalmic artery or the junction between the ophthalmic artery and the internal carotid artery of the subject.

In some embodiments, the step of accessing the terminal branch of the ophthalmic artery through the skin of the head of the subject via the first device includes accessing the terminal branch via a cut-down procedure.

In some embodiments, the step of performing the atherectomy includes performing the atherectomy via an atherectomy element including a basket-shaped structure.

In another embodiment, there is provided another method which may include: accessing a terminal branch of an ophthalmic artery through a skin of a head of a subject via a first device; positioning at least a portion of the first device or a second device within the ophthalmic artery distal of an area to be treated within the ophthalmic artery, wherein the first device includes an atherectomy element; and treating tissue of the ophthalmic artery by withdrawing the at least the portion of the at least one of the first device or the second device toward the area to be treated to increase vascular flow through the ophthalmic artery.

In some embodiments, the step of withdrawing the portion of the at least one of the first device or the second device causes tissue debris.

In some embodiments, the method may also include capturing the tissue debris and removing the tissue debris from the subject.

In some embodiments, the method may also include performing an angioplasty on the area to be treated.

In some embodiments, the step of accessing the terminal branch of the ophthalmic artery through the skin of the head of the subject includes accessing one of a supraorbital artery, a dorsal nasal artery, or a supratrochlear artery of the subject via the first device.

In some embodiments, the method may also include measuring a blood flow rate in the ophthalmic artery.

In some embodiments, the step of treating the tissue of the ophthalmic artery includes treating an eye disease, wherein treating the eye disease comprises treating macular degeneration.

In another embodiment, there is provided yet another method which may include: positioning a first device in a terminal branch of an ophthalmic artery through a skin of a head of a subject, wherein positioning the first device in the terminal branch of the ophthalmic artery through the skin includes positioning the first device in one of a supraorbital artery, dorsal nasal, or a supratrochlear artery; positioning at least a portion of the first device or a second device within the ophthalmic artery distal of an area to be treated within the ophthalmic artery, wherein the at least the portion of the first device or the second device includes an atherectomy element; and performing an atherectomy on the area to be treated by moving the atherectomy element relative to the area to increase vascular flow through the ophthalmic artery.

In some embodiments, the method may also include performing an angioplasty on the area.

In some embodiments, the step of moving the atherectomy element relative to the area to be treated causes tissue debris.

In some embodiments, the method may also include capturing the tissue debris and removing the tissue debris from the subject.

In some embodiments, the method may also include measuring a blood flow rate in the ophthalmic artery.

The present disclosure relates to, for example, devices and methods for percutaneous frontal access via the supratrochlear or the supraorbital artery for atherectomy treatment of the ophthalmic artery and vascular structures in the rear of the eye, including treatment for the symptoms related to WAMD by removal of stenosis of the OA, thereby restoring normal, or near normal, blood flow to the rear of the eye.

In one embodiment, there is provided a method for treating occlusion of the ocular blood vessels. The method may include: frontally accessing a supratrochlear, nasal dorsal, or supraorbital artery of a patient; deploying, into the supratrochlear or supraorbital artery, nasal dorsal, or supraorbital artery an atherectomy device in a retrograde approach, said atherectomy device comprising a tapered corewire ranging in diameter from about 0.19 mm to about 0.88 mm, the corewire being disposed within a delivery sheath, said corewire having a material cutting element at or near a distal end, said corewire having an integral inflatable balloon section at the distal end as a protective element, and said corewire having an atraumatic tip; guiding the atherectomy device to a lesion site in the ophthalmic artery; and performing an atherectomy of the lesion to remove plaque material.

In some embodiments, the step of frontally accessing the supratrochlear or supraorbital artery of the patient may comprise a surgical cut down procedure.

In some embodiments, the step of frontally accessing the supratrochlear or the supraorbital artery of the patient may comprise percutaneously accessing the supratrochlear, nasal dorsal, or the supraorbital artery of the patient.

In some embodiments, the step of guiding the atherectomy device may include use of a non-invasive real-time imaging methodology.

In some embodiments, the real-time imaging methodology may be selected from the group consisting of fluoroscopy, near-IR fluorescence imaging, optical coherence tomography (OCT), magnetic resonance imaging, ultrasound imaging, color doppler (ultrasound) imaging, angiography using visualization media, and optical doppler tomography, which uses high-resolution OCT with laser doppler.

In some embodiments, the use of distal protection may be provided for the internal carotid artery (ICA).

In some embodiments, the use of distal protection may be provided for the OA.

In some embodiments, the method may further comprise the step of removing debris by aspiration while the atherectomy device is in the OA.

In some embodiments, the atherectomy device may be constructed of a solid corewire with a mounted atherectomy and distal protection device.

In some embodiments, the atherectomy device may be constructed of a balloon designed to inflate such that contact with target anatomy is achieved.

In some embodiments, the balloon may have external materials affixed directly to the balloon surface to facilitate an atherectomy.

In some embodiments, the balloon may have external emboli protection.

In some embodiments, the balloon may be a balloon mounted on a polymer catheter.

In some embodiments, the balloon may be a balloon mounted on a solid corewire.

In some embodiments, the balloon may be mounted on a hypotube.

In some embodiments, a device may be provided for the removal of debris by aspiration.

In some embodiments, the atherectomy device may be made of materials selected from nitinol, stainless steel, or other materials suitable with intravascular medical devices.

In some embodiments, the atherectomy device may be configured as a single hypotube cut to contain a combination atherectomy device and distal protection device.

In some embodiments, the method further may include diagnosing one or more symptoms of WAMD or dry age-related macular degeneration (DAMD) in the patient.

In some embodiments, a surgical kit may be provided containing an atherectomy kit configured and sized for percutaneous access to an ocular blood vessel, and instructions may be provided for performing a frontal access deployment through the supratrochlear or supraorbital artery.

In some embodiments, a kit may be provided comprising a container of fluoroscein dye or another radiopaque solution.

### BRIEF DESCRIPTION OF THE FIGURES

FIG. 1 is an anatomical schematic drawing of an eye.

FIG. 2 is an anatomical schematic drawing of a central retinal artery (CRA) within an optic nerve sheath.

FIG. 3 is a schematic diagram illustrating branches and cross connections of ocular blood vessels.

FIG. 4 is an anatomical schematic drawing of frontal eye anatomy, including a supratrochlear (ST) artery of an eye.

FIG. 5A is an anatomical schematic diagram showing the ST artery, a supraorbital (SO) artery, and a dorsal nasal (DN) artery of the eye.

FIG. 5B is an anatomical schematic diagram illustrating access arteries of an ophthalmic artery (OA).

FIG. 6 is an illustration of a microcannulation device deployed into a selected access artery, according to embodiments of the present disclosure.

FIGS. 7A-7C illustrate a process of performing frontal access to the OA using a cut-down technique, according to an embodiment of the present disclosure.

FIG. 8 is a schematic drawing of the cut-down technique used to expose the SO artery, according to an embodiment of the present disclosure.

FIG. 9 is a schematic drawing of an interventional device, according to an embodiment of the present disclosure.

FIG. 10 is a schematic drawing of an interventional device including an aspiration core and a particle filter, according to an embodiment of the present disclosure.

FIGS. 11A-11C are schematic diagrams of an interventional device having a reaming mechanism, according to an embodiment of the present disclosure.

FIGS. 12A and 12B are semi-transparent perspective side views of an interventional device having an aspirational core, according to an embodiment of the present disclosure.

FIG. 13 is a semi-transparent perspective side view of an interventional device, according to an embodiment of the present disclosure.

FIG. 14A is a schematic drawing showing a corewire provided in an interventional device, according to an embodiment of the present disclosure.

FIG. 14B is a schematic drawing showing a distal tip segment of a corewire of an interventional device, according to an embodiment of the present disclosure.

FIG. 15 is a side view a corewire of an interventional device, according to an embodiment of the present disclosure.

FIGS. 16A and 16B are side views of an interventional device, according to an embodiment of the present disclosure.

FIG. 16C is a side view of an interventional device, according to an embodiment of the present disclosure.

FIGS. 17A and 17B are side views of an interventional device, according to an embodiment of the present disclosure.

FIGS. 18A-18C include a series of drawings of a hypotube atherectomy corewire and an expanded atherectomy balloon with a distal protection element, according to an embodiment of the present disclosure.

FIGS. 19A and 19B are side views of a multicomponent apparatus, according to an embodiment of the present disclosure.

FIGS. 20A and 20B are side views of an interventional device, according to an embodiment of the present disclosure, before and after placement within target anatomy.

FIGS. 21A-21C are a series of drawings of variations in balloon distal elements, according to an embodiment of the present disclosure.

FIGS. 22A-22C are a series of line drawings of use of a catheter to access an OA and straight and shaped guide wires, according to an embodiment of the present disclosure.

### DETAILED DESCRIPTION

The present disclosure relates to devices and methods of performing an ophthalmic atherectomy for treatment of occlusion of an ophthalmic artery (OA) of an eye. Without being limited to any specific theory, the present disclosure is based on the premise that the primary causative effect for Wet Age-Related Macular Degeneration (WAMD), glaucoma, and diabetic retinopathy is occlusion of the OA, such that normal blood flow is restricted (ischemia) to the rear of the eye. As a result of this ischemia, hypoxia (resulting in neovascularization) is induced in these structures, and vision eventually devolves into a dysfunctional retina (WAMD). From this premise, we have identified a method and design that may be used to provide a treatment methodology for WAMD. Several variations are detailed later in this specification.

### Vascular Supply to the Eye

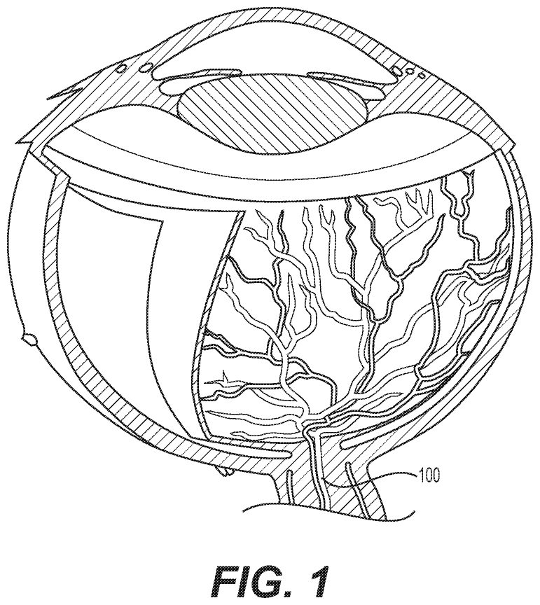

FIGS. 1-5B provide detailed background information on the vasculature and structures of the eye. The OA 100, which is the first branch of the internal carotid artery (ICA) 102, provides the majority of the eye's blood supply. The OA 100 enters the orbit through the optic canal and runs, usually inferolaterally, to the optic nerve. After traveling nasally and anteriorly, the OA 100 continues superiorly to the optic nerve, where it branches. Some of these branches extend to the ocular muscles, namely, the central retinal artery (CRA) and the posterior ciliary arteries (PCAs).

There are commonly 2 or 3 PCA trunks. The PCA trunks each have ten to twenty shorter PCA branches before connecting to the sclera.

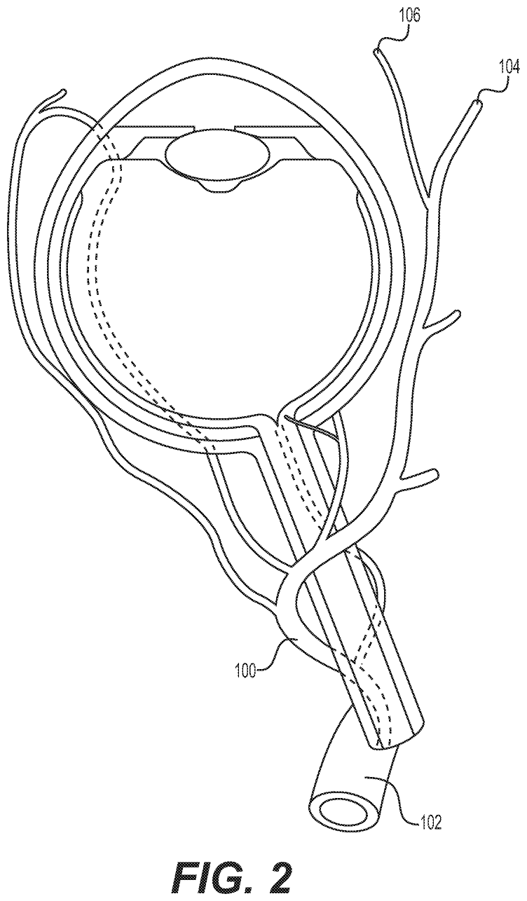

As shown in FIG. 2, the CRA travels through the optic nerve sheath about 10 mm posterior to the eyeball, usually, and over the top (superiorly) of the optic nerve. However, it is noted that there is a higher predisposition to age-related macular degeneration (AMD) (e.g., WAMD, DAMD) in patients where the CRA travels through the sheath of, and under, the optic nerve (inferiorly). The lumen of the part of the CRA travelling through the optic nerve sheath is about 200 μm in diameter. The CRA divides into four branches: the superior nasal retinal artery, the inferior nasal retinal artery, the superior temporal retinal artery, and the inferior temporal retinal artery. Nasal refers to the location below the eye, and temporal refers to the location above the eye.

The retinal arteries supply the inner two-thirds of the retina with its blood supply, and the ciliary arteries supply the outer one-third of the retina with its blood supply. The retinal arteries, unlike other arteries, do not possess an internal elastic lamina nor a continuous muscular coat. The lumen of the retinal arteries has a diameter of about 100 μm.

The OA 100 also has two other main branches in addition to the ocular branch described above, namely, the orbital branch and the extraorbital branch. The orbital branch includes the lacrimal artery, extraocular muscle arteries, and arteries that supply orbital tissue. The extraorbital branch includes the anterior and posterior ethmoidal arteries, the supraorbital artery 108, the medial palpebral artery, and the terminal ophthalmic artery branches—the dorsal nasal artery 104 and the supratrochlear artery 106.

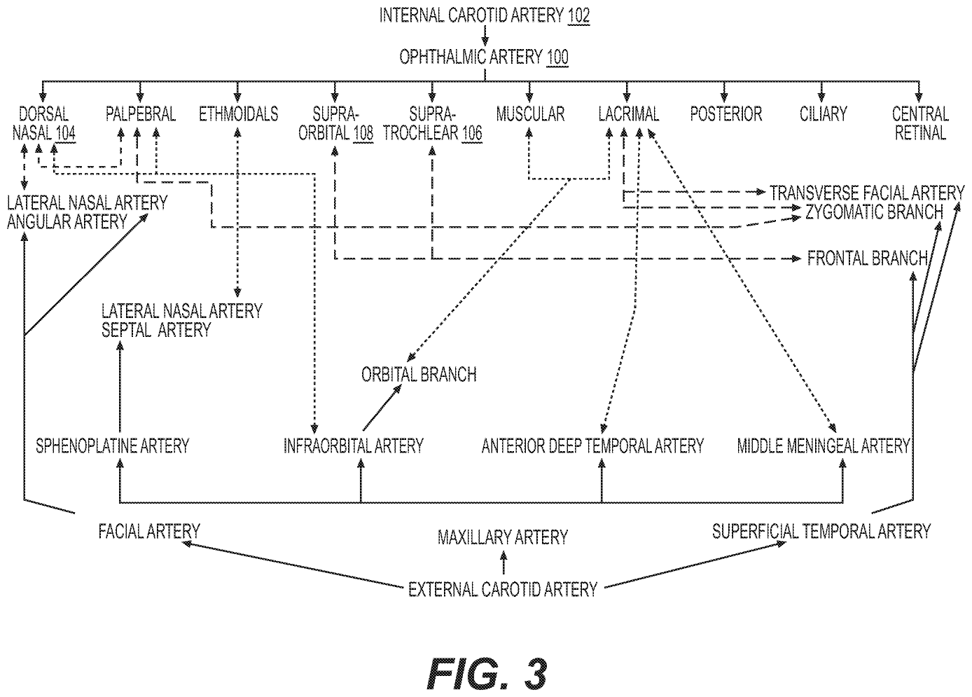

FIG. 3 shows that the arteries of the eye are also known to have numerous anastomoses. An anastomosis is where branches of the ocular blood vessels reconnect with other branches. These cross connections can also provide access for therapeutic devices and treatments.

### Frontal Access Technique

Referring now to FIG. 4, the supratrochlear artery 106 is illustrated. Starting from the ICA 102 and proceeding in an antegrade sequence, FIG. 4 shows the OA 100 branching off with the lacrimal artery branching at the first bend. Closer to the eye, the OA 100 divides to form the supratrochlear (ST) artery 106 and the supraorbital (SO) artery 108, which then supply various eye structures via lesser branches before exiting the skull at the supraorbital foramen. The dorsal nasal (DN) artery 104 is also shown branching off from the subtrochlear artery 106.

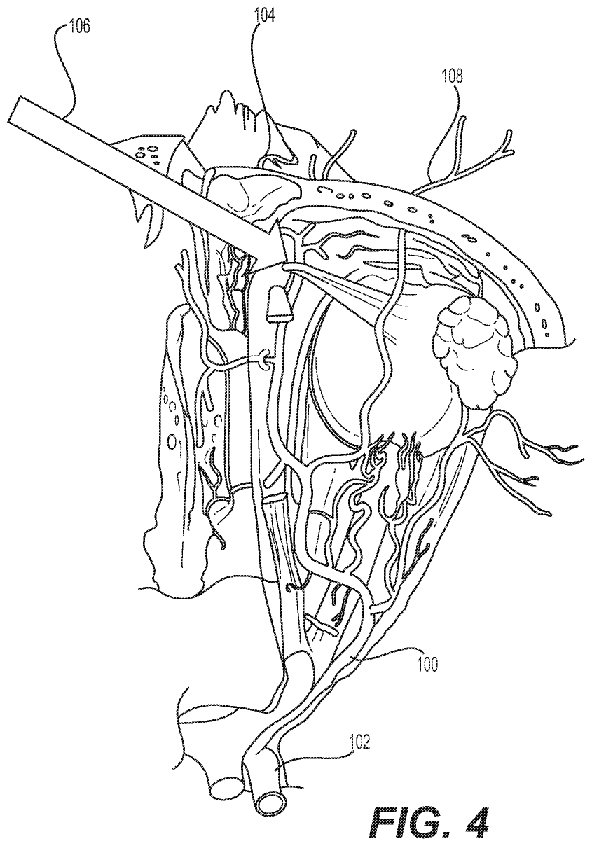

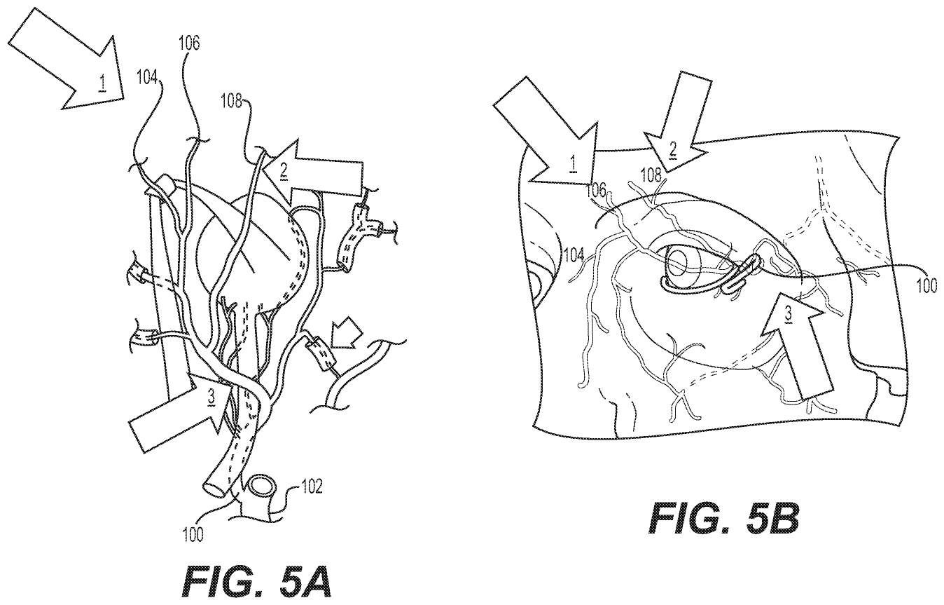

FIG. 5A is another diagram of the ocular anatomy and shows the ST artery 106, the SO artery 108, and the DN artery 104 as access vessels for accessing the OA 100. The branching occurs just anterior to the loci where the OA 100 traverses first under the optic nerve and then over the optic nerve. FIG. 5A also shows that the lacrimal artery may be used as an access point if the arterial plaque within the OA 100 occurs between the lacrimal branch and the ICA 102. FIG. 5B is an external anatomical illustration showing how cannulating the ST artery 106, the DN artery 104, and/or the SO artery 108 provides access to the OA 100 for treatment.

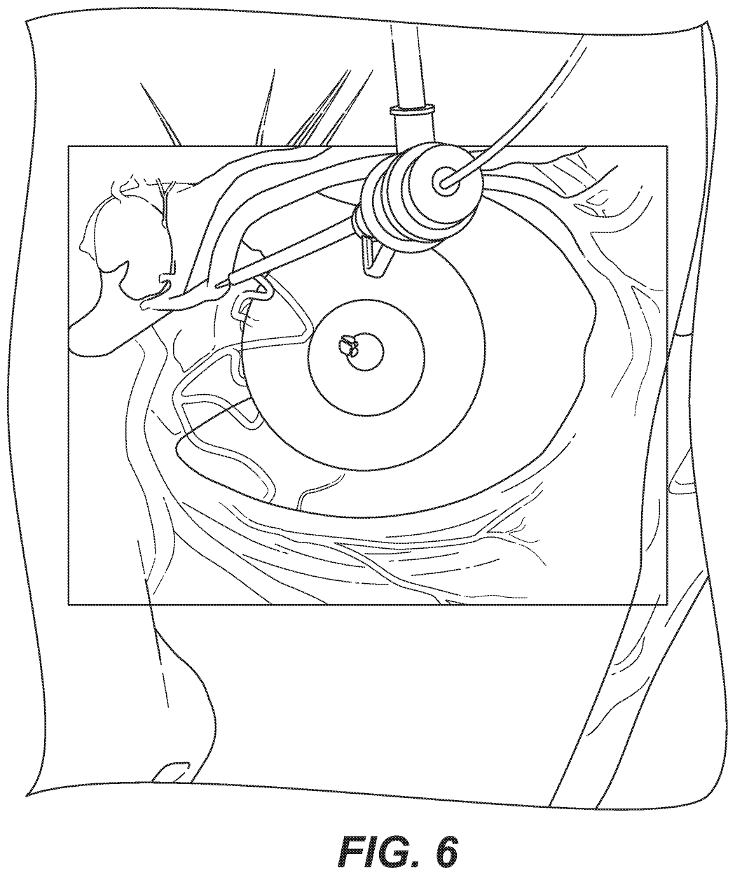

FIG. 6 is a graphic illustration showing a microcannulation device, as described herein, that has been deployed into either the ST artery 106, the DN artery 104, or the SO artery 108. Frontal access via the ST artery 106, the DN artery 104, or the SO artery 108 for atherectomy treatment of an occluded OA 100 or other vascular structures in the rear of the eye provides the restoration of normal, or near normal, blood flow to the rear of the eye. Accessing from the front in a retrograde fashion significantly simplifies the procedure as compared to a vascular atherectomy that might approach from the femoral artery, the subclavian artery, or the internal carotid artery 102.

The basic steps of this method include frontally accessing one of an ST artery 106, a DN artery 104, or an SO artery 108 (e.g., a target artery) of a patient, and deploying, into the one of the ST artery 106, the DN artery 104, or the SO artery 108, an atherectomy device in a retrograde approach. The atherectomy device may comprise a tapered corewire ranging in diameter from about 0.19 mm to about 0.88 mm, and may be disposed within a delivery sheath. In addition, the corewire may have a material cutting element at or near a distal end, an integral inflatable balloon section at the distal end as a protective element, and an atraumatic tip. In addition, the method may include the steps of guiding the atherectomy device to a lesion site in the OA 100, and performing an atherectomy of the lesion to remove plaque material.

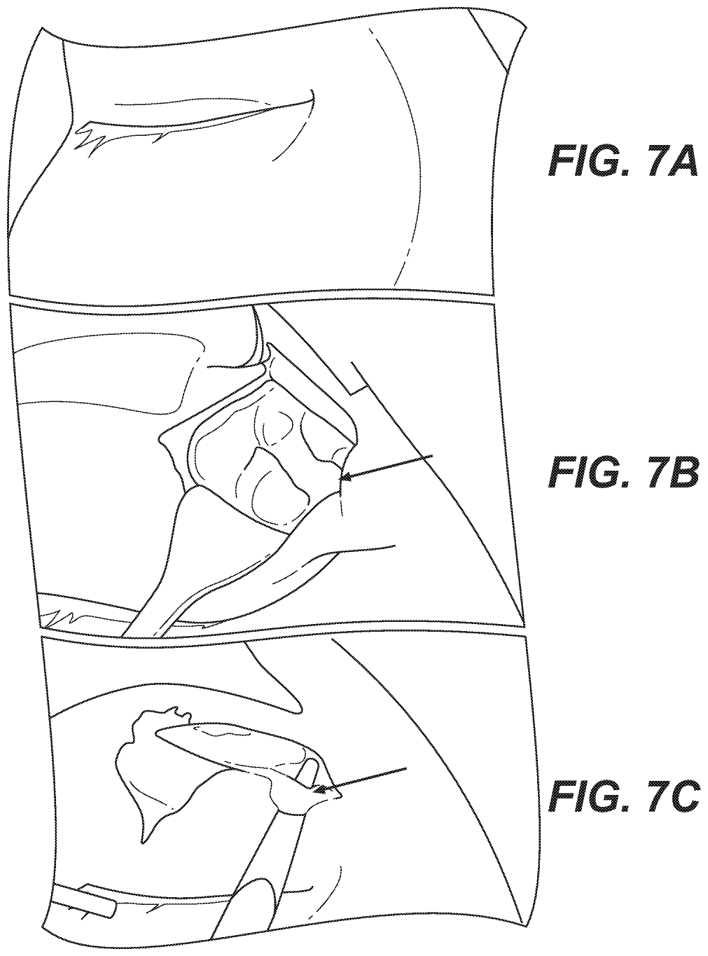

FIGS. 7A-7C show that frontal access may be performed using a cut-down technique where the target artery is accessed by making surgical incisions to expose the artery. Another technique contemplated as within the scope of the present disclosure includes using a percutaneous technique whereby the target artery is accessed using a needle, and the atherectomy device is then deployed into the target artery. A guide wire may be used to facilitate removal of the needle and insertion of the microcannulation device.

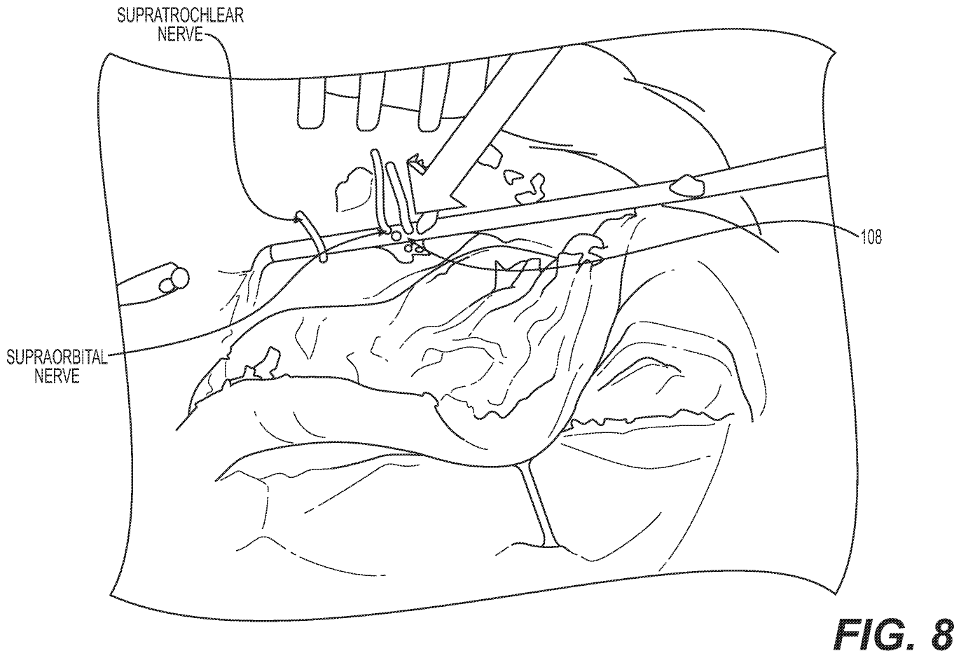

FIG. 8 is a representation of a cut-down technique to expose the SO artery 108. FIG. 8 also shows the proximity of the supratrochlear nerve and the supraorbital nerve.

### Technologies for Measuring Blood Flow to the Eye

Technologies for measuring ocular flow may include color doppler (ultrasound) imaging, angiography using visualization media, such as fluorescein, and optical doppler tomography, which uses high resolution optical coherence tomography (OCT) with laser doppler to measure the flow in retinal arteries in real time.

### Interventional Device

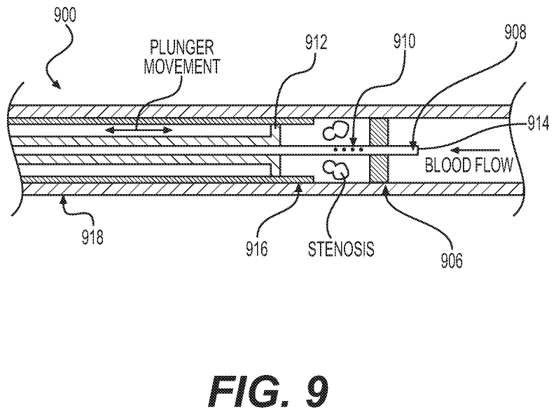

Referring now to FIG. 9 and the subsequent figures, an interventional device 900 will described. The interventional device 900 may be designed to gain access to, and deliver direct mechanical and/or drug therapy, to a specific location of the anatomy (e.g., target artery). While the following examples specifically detail the necessary components for a particular ophthalmic artery (OA) application (e.g., where the target artery is the OA), this technology may be used in any anatomical location in which removal of material is desired in a luminal environment. This environment may be vascular or not, and may be used in any tubal, luminal, or other similar anatomical structure where removal of material is desired. As such, the disclosed interventional devices can be scaled, modified, or constructed such that it can provide therapy for a specific luminal anatomical location/need.

The design of a general interventional device may be based on a central wire, a hypotube, a coil, a balloon, or a combination thereof. The interventional device may be made of stainless steel, nitinol, polymer, other materials, or a combination thereof, and may be designed to accommodate specific approaches (e.g., carotid, subclavian, femoral, endoscopic, or laparoscopic). For the example given, entrance into the body is provided by a vascular access element, which may be readily available, or may be designed specifically for use with the interventional device (e.g., a catheter sheath introducer or an equivalent device). The interventional device may fit within a sheath, which may be designed to provide a protective element for the device, as well as to prevent vessel trauma during delivery to the target anatomy. The distal portion of the interventional device may include the ability to provide distal protection in the OA, as well as an element to provide diametric interference. This area of diametric interference may be designed to interface with the target vessel segment (e.g., a lesion site), such that specific and deliberate manipulation provides for the ability to selectively remove material from the lesion site. The diametric interference element also may provide for the ability to compress, such that it fits within the device sheath to provide a minimal diametric dimension. This diametric portion also may be referred to as an interventional element.

Once the interventional device is placed at the target anatomy, the interventional element may be positioned such that it is located outside of the sheath (e.g., extends distally of the sheath) and it may conformably fit the inner diameter of the target anatomy. The interventional element also may contain a design element that allows for plaque removal when manipulated in a specific manner, such as, for example, manual rotation, manual push/pull, mechanical rotation, mechanical push/pull, or a combination of some or all of those manners of manipulation. Once material removal is complete, the interventional element may be pulled into the sheath, along with the distal protection portion (if so equipped) of the device, and the entire assembly removed. It is also possible to remove the interventional element for cleaning and to replace and continue.

### Interventional Device—Common Device Aspects and Elements

Common aspects and elements of an interventional device (e.g., common to the various interventional devices described herein) may include an ability to visualize under fluoroscopy; ST or SO access; a distal protection element in the internal carotid artery (ICA); a protection element in the ophthalmic artery (OA); working in OA diameter ranges between 0.7 to 1.4 mm—derived by atmospheric pressure applied to the conformal element; and a working length for the OA being estimated to be about 15 inches, although additional working lengths may be used. Other aspects of the interventional device may include approaches other than ST or SO, an ability to remove material from the OA and transport the removed material out of the vasculature, and an ability to induce retrograde flow, either continuously or on demand, for specific time periods. Further, the interventional device may use a guiding catheter (GC) to cannulate the OA from the ST or the SO, or a combination of GC features with a sheath to have an ‘all in one’ device.

As shown in FIG. 9, an interventional device 900, such as an atherectomy device, may include a sheath 916 that is pulled back such that a cutting element (not shown in FIG. 9) and a distal protection element (e.g., occlusion balloon) 906 may be deployed. Aspiration may be utilized and is accomplished by either flushing or aspirating using alternate longitudinal channels of a delivery wire 908. For example, a first lumen extending from a proximal end toward a distal end of delivery wire 908 may delivery fluid or other material to the distal end of delivery wire 908 (e.g., flush) while a second lumen extending from the proximal end toward the distal end of delivery wire 908 may apply suction (e.g., aspirate). Alternatively, a single lumen of delivery wire 908 may alternatingly provide flushing and aspiration capabilities for interventional device 900. Once the procedure is complete, the elements of the interventional device 900 may be withdrawn back into the catheter sheath 916, and interventional device 900 may be then safely withdrawn from the anatomy.

Generally, the overall length of the interventional device 900 may be determined based on a selected anatomical location and approach. In an example, for use within the OA, an overall length of about 160 cm or about 15.00 inches for the device 900 would be used in conjunction with an appropriately designed delivery sheath. The maximum overall diameter of the delivery sheath 916 would be in the 1.0 mm range (after inflation), with the cutting element (not shown) and the distal protection element 906 offering a conformal fit capability in a deployed range of between 0.7 mm to 1.4 mm, as dictated by the specific dimensions of the OA and the lesion site. These overall length and diametric dimensions may be adjusted based on specific applications (e.g., specific patient vasculature dimensions), and any such adjustment is contemplated as within the scope of the present disclosure. In addition, the specific material composition, formulation, and manufacturing parameters of material used may be refined to address the specific application, and any such refinement is contemplated as within the scope of the present disclosure. This dimensional information applies to all of the designs disclosed. In one example, the lesion crossing profile of this device 900 is less than 0.2 mm. A range of appropriate profile dimensions is contemplated as within the scope of the present disclosure.

### Atherectomy Literature

The following documents illustrate and describe various features of atherectomy devices and methods: U.S. Pat. Nos. 8,439,937, 5,314,438, 6,494,890, 5,409,454, 4,898,575, 4,857,045, 4,794,931, 5,000,185, 5,313,949, 5,507,292, 4,950,277, 4,986,807, 5,019,088, 4,894,051, 4,957,482, 4,979,939, 5,007,896, 5,024,651, 5,135,531, 5,087,265, 5,318,576, 5,366,464, 5,402,790, Mazur et al., Catheterization and Cardiovascular Diagnosis 31:79-84 (1994), U.S. Pats. No. 4,886,061, and 5,100,425. Each of the above references is incorporated herein in its entirety.

### Interventional Device—Specific Examples: Frontal facial

As noted above, FIG. 9 shows the interventional device 900 according to an embodiment of the present disclosure, and having an aspiration core as the delivery wire (also referred to herein as a central corewire or a central member) 908. The design is based on a solid metallic corewire with an integrated aspiration capability. The interventional device 900 may include the central corewire 908, flushing holes 910, a delivery sheath, a plunger 912, a distal protection element/occlusion balloon 906, an atraumatic tip 914, and a catheter sheath 916.

FIG. 9 depicts the device 900 with the catheter sheath 916 covering a portion of the central corewire 908, and the plunger 912 and the distal protection element 906 (e.g., occlusion balloon) both being mounted on the central corewire 908. The tip of the device 900 contains the atraumatic tip 914 to aid in placement of the device 900 with an artery 918.

In the device of FIG. 9, catheter sheath 916 has been pulled back so as to permit deployment of the cutting element (not shown) and the distal protection element 906. Aspiration is accomplished by either flushing and aspirating using alternate longitudinal channels of the central corewire 908, or by a combination of use of longitudinal channels and the catheter sheath 916, one for flushing and the other for aspiration, as noted above. Once the procedure is complete, the elements of the device 900 are withdrawn back into the catheter sheath 916, and the device 900 is then safely withdrawn from the anatomy.

### Interventional Device—Specific Examples: Frontal Facial With Filter

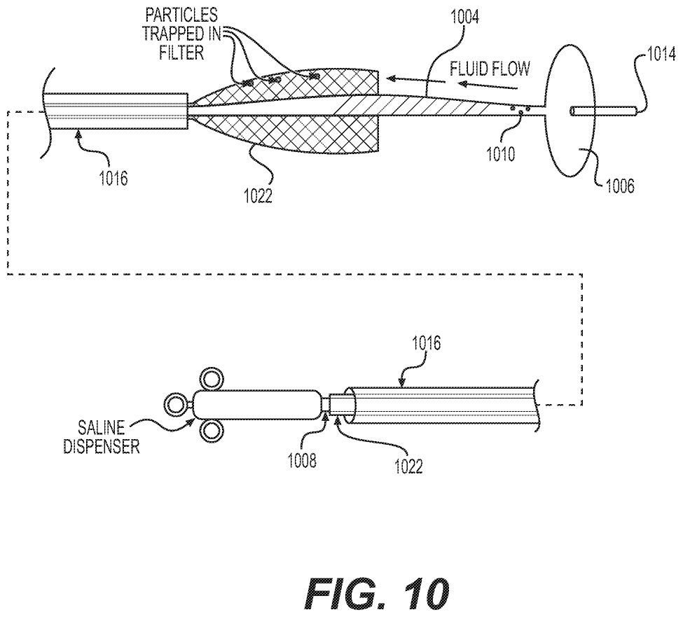

FIG. 10 shows an interventional device 1000 according to an embodiment of the present disclosure, the device 1000 having an aspiration corewire 1008 and a particle filter 1022 for trapping dislodged material. The design is based on a solid metallic corewire with integrated aspiration capability. The device 1000 includes a central corewire 1008, a reaming or cutting element 1004, flushing/aspiration holes 1010, a nickel-titanium (NiTi) mesh filter 1022, a distal protection element/occlusion balloon 1006, an atraumatic tip 1014, and a movable guiding catheter sheath (guide sheath) 1016.

FIG. 10 depicts the device 1000 with the guiding catheter sheath 1016 covering at least a portion of the central member 1008. The central member 1008 having the reaming element 1004, the mesh filter 1022, and the distal protection/balloon element 1006 each mounted thereon. The tip of the device 1000 contains the atraumatic tip 1014 to aid in placement of the device 1000.

FIG. 10 depicts the guiding catheter sheath 1016 pulled back (e.g., proximally withdrawn), and the reaming element 1004 and the distal protection element 1006 both in a deployed position. Aspiration is accomplished by either flushing/aspirating using alternate longitudinal channels of the corewire 1008, or by a combined use of longitudinal channels and the guiding catheter sheath 1016, one for flushing and the other for aspiration. Once the procedure is complete, the elements of the device 1000 are withdrawn back into the guiding catheter sheath 1016, and the device 1000 is then safely withdrawn from the anatomy. FIG. 10 also shows that the guide sheath 1016 may optionally be a moveable guiding catheter sheath 1016 to facilitate the use of additional positions during aspiration.

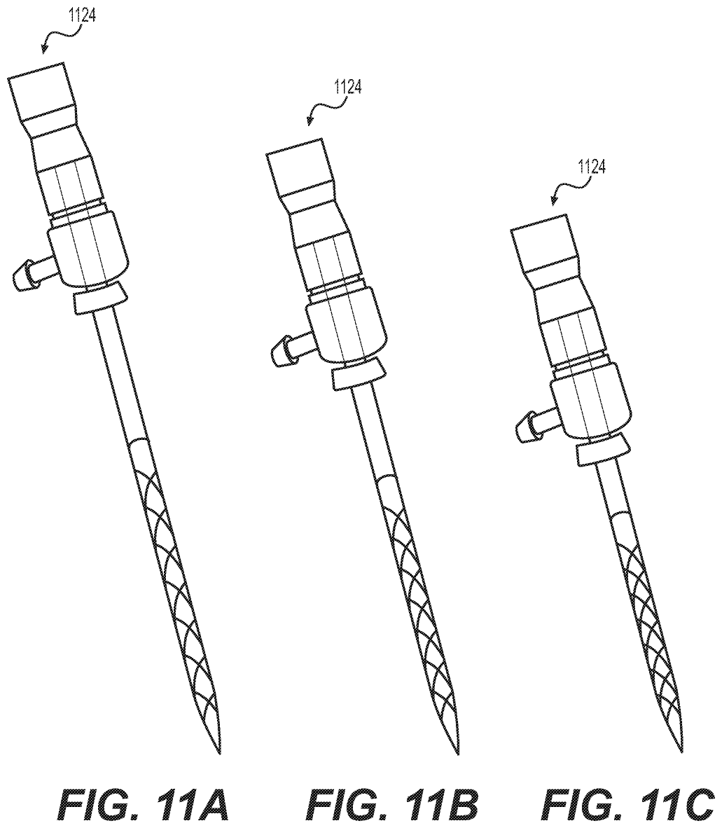

FIGS. 11A-11C illustrate an exemplary alternate reaming mechanism 1122 that uses a self-adjusting file. This reaming mechanism 1122 allows the compressed reaming element 1122 to be delivered via a delivery sheath and then, upon deploying out of the delivery sheath, expands to form a plaque removing metallic matrix. Though a distal end of the reaming mechanism 1122 is illustrated as including a pointed/angled tip, in some arrangements, the distal end may be blunt or flat so as not to form a pointed tip.

### Interventional Device—Specific Examples: Solid Corewire based

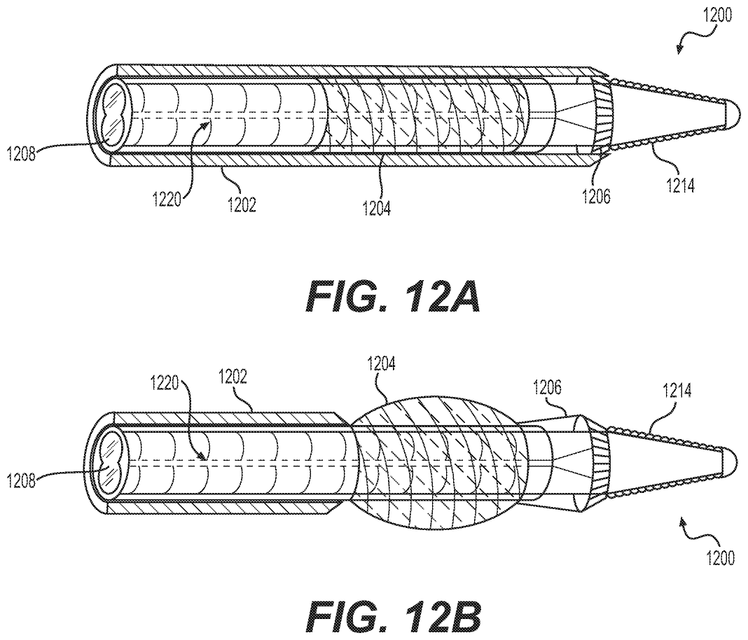

FIGS. 12A and 12B illustrate an interventional device 1200 according to an embodiment of the present disclosure, the device 1200 having an aspiration corewire 1208. The design is based on a solid metallic corewire with integrated aspiration capability. The device 1200 may include the central corewire 1208, longitudinal indentations 1220, a delivery sheath 1202, a cutting element 1204, a distal protection element 1206, and an atraumatic tip 1214.

FIG. 12A depicts the device 1200 with the delivery sheath 1202 covering the cutting element 1204 and the distal protection element 1206, which are both mounted on the central corewire 1208. The tip of the device 1200 contains the atraumatic tip 1214 to aid in placement of the device 1200.

FIG. 12B depicts the delivery sheath 1202 pulled back, and the cutting element 1204 and the distal protection element 1206 both in a deployed position. Aspiration is accomplished by either flushing and aspirating using alternate longitudinal channels defined (at least in part by) indentations 1220 of the central corewire 1208, or by a combination use of longitudinal channels 1220 and the delivery sheath 1202, one for flushing and the other for aspiration. Once the procedure is complete, the elements of the device 1200 are withdrawn back into the delivery sheath 1202 and are positioned as shown in FIG. 12A. The device 1200 is then safely withdrawn from the anatomy.

Generally, the overall length of the device 1200 is optimized for the anatomical location and approach. In an example, for use within the OA, an overall length of about 160 cm or about 15.00 inches for the device 1200 would be used in conjunction with an appropriately designed sheath 1202. The maximum overall diameter of the sheath 1202 would be in the 1.0 mm range (after inflation), with the cutting element 1204 and the distal protection element 1206 offering a conformal fit capability in the deployed range of between 0.7 mm to 1.4 mm, as dictated by the specific dimensions of the OA and the lesion site. These overall length and diametric dimensions may be adjusted based on the specific applications, and any such adjustment is contemplated as within the scope of the present disclosure. In addition, the specific material composition, formulation, and manufacturing parameters of material used would be refined to address the specific application, and any such refinement is contemplated as within the scope of the present disclosure. This dimensional information applies to all of the designs disclosed. In one example, the lesion crossing profile of this device 1200 is less than 0.2 mm. A range of appropriate profile dimensions is contemplated as within the scope of the present disclosure.

### Interventional Device—Specific Examples: Plain core—Non Aspiration Core

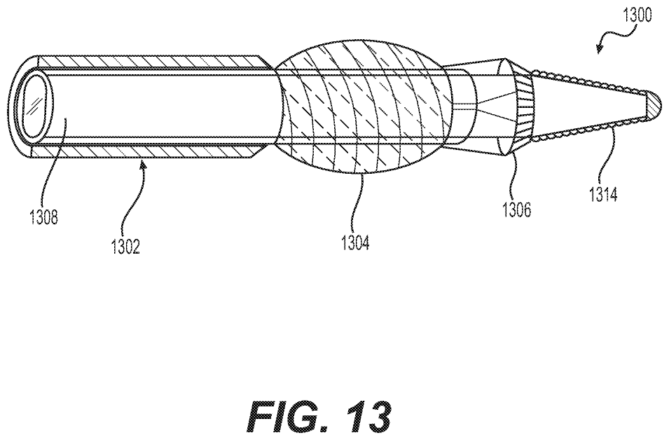

The design of an interventional device 1300 shown in FIG. 13 is based on the device 1300 having a solid corewire 1308, and does not have specific aspiration capability. The device 1300 includes the central corewire 1308, a delivery sheath 1302, a cutting element 1304, a distal protection element 1306, and an atraumatic tip 1314.

This device 1300 shown in FIG. 13 is essentially the same as the device 1200 shown in FIGS. 12A and 12B, with the exception that the corewire 1308 is not designed to facilitate aspiration. The remaining elements of the device 1300 are essentially similar to the device 1200 shown in FIGS. 12A and 12B. FIG. 13 shows the delivery sheath 1302 pulled back, and the cutting element 1304 and the distal protection element 1306 both in a deployed position. Once the procedure is complete, elements of the device 1300 are withdrawn back into the delivery sheath 1302 and are positioned in a similar fashion as seen in the device 1200 shown in FIG. 12A. The device 1300 is then safely withdrawn from the anatomy.

It should also be noted that the corewire based design may include elements that are much simpler in design than those shown in the figures described above. These designs could include a corewire with a specific drawn profile that is inserted into the anatomy, such that movement of the corewire would allow an interface between the profile of the corewire and the anatomy to facilitate lesion material removal. These particular designs could include a straight ‘as drawn’ wire, an ‘as drawn’ wire with a twist, and a selective combination of the two.

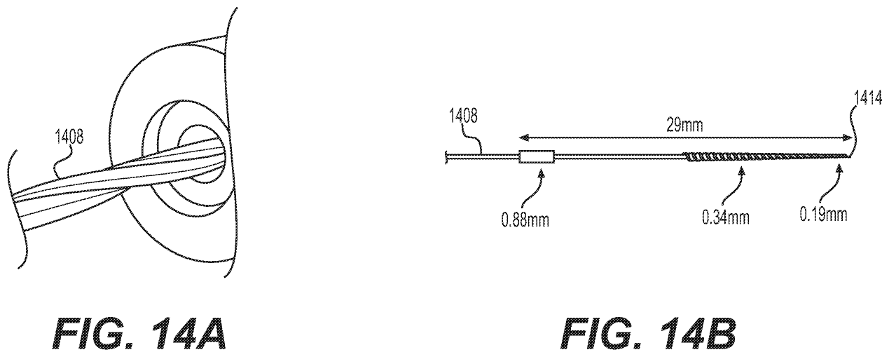

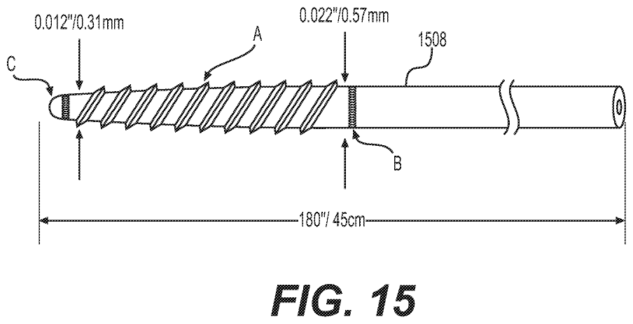

FIGS. 14A and 14B depict a drawn corewire 1408 with a twist and a distal tip 1414 segment of the corewire 1408 according to one embodiment of the present disclosure. FIG. 15 depicts another corewire 1508 according to another embodiment of the present disclosure.

### Interventional Device—Hypotube Based

### Integral Elements

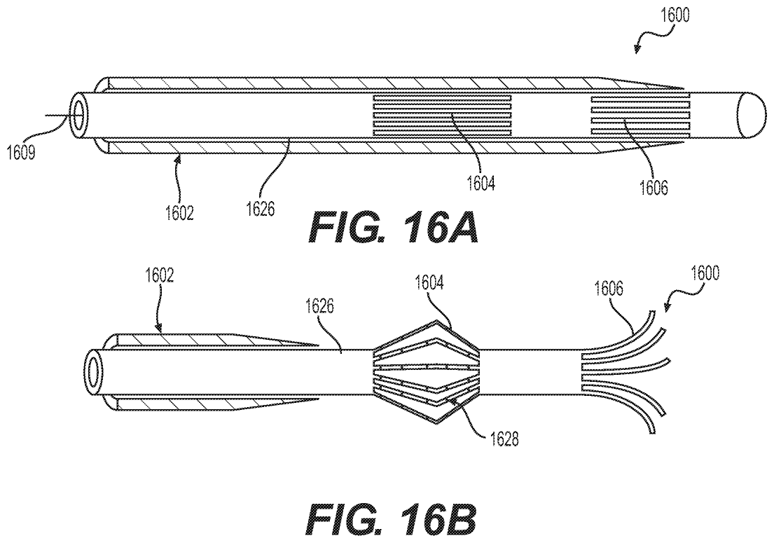

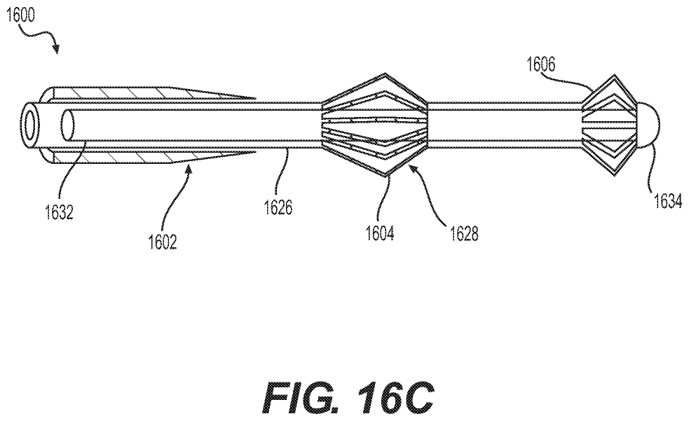

FIGS. 16A and 16B show an interventional device 1600 with a corewire design being based on a hollow metallic tube (hypotube). Aspiration capability is not detailed in this series of drawings, but may be possible with the addition of a central flush source. The device 1600 may include a central guidewire 1609, a delivery sheath 1602, a hollow tube (hypotube) 1626, a cutting element 1604, a distal protection element 1606, and abrasives 1628.

FIG. 16A depicts the device 1600 with the delivery sheath 1602 covering the cutting element 1604 and the distal protection element 1606, which are both cut from the hypotube 1626, and as such, are integral to the hypotube 1626.

In an alternative embodiment (not shown), the cutting element 1604 and the distal protection element 1606 are mounted on the hypotube 1626. In another embodiment, an additional element may be positioned in the lumen of the hypotube after removal of the guidewire 1609. This element would serve to deliver fluid for flushing. In this example, aspiration could be accomplished by applying suction to a proximal end of the hypotube 1626, such that fluid is removed as well as debris while flushing is activated.

The guidewire 1609 may extend down (e.g., along, through) an inner lumen of the hypotube 1626 to provide a means for navigating the anatomy. Upon placement of device 1600 within the target anatomy, the guidewire 1609 may be removed, and the sheath 1602 pulled back, deploying the cutting element 1604 and the distal protection element 1606. Deployment of the cutting element 1604 and the distal protection element 1606 may be controlled by selective manufacturing processes, which preferentially ‘train’ the elements to move in a certain fashion, such that they exhibit a condition known as ‘shape memory’. This shape memory is exhibited by the hypotube 1626 when it is in an unrestrained position. The abrasives 1628, which are mounted, coated, or integral with the cutting element 1604, may be designed to facilitate material removal and shaping of the lesion.

FIG. 16B depicts the delivery sheath 1602 pulled back, and the cutting element 1604 and the distal protection element 1606 both in a deployed position. Once the procedure is complete, the elements of the device 1600 are withdrawn back into the delivery sheath 1602 and are positioned as in the device 1600 shown in FIG. 16A. The device 1600 can then be safely withdrawn from the anatomy.

FIG. 16C depicts an alternative embodiment of the hypotube 1626 design. In this alternative embodiment, the elements of the device 1600 of FIG. 16C are similar to corresponding elements illustrated in FIGS. 16A and 16B, with the exception of a moveable internal corewire 1632, which is joined with an inner distal tip 1634 of the hypotube 1626, such that longitudinal movement of the corewire 1632 may serve to either expand or compress the cutting element 1604 and the distal protection element 1606. When the procedure is complete, removal of this device 1600 would be accomplished in a similar fashion as described with reference to FIG. 16B, above.

### Interventional Device—Polymer Based Tube

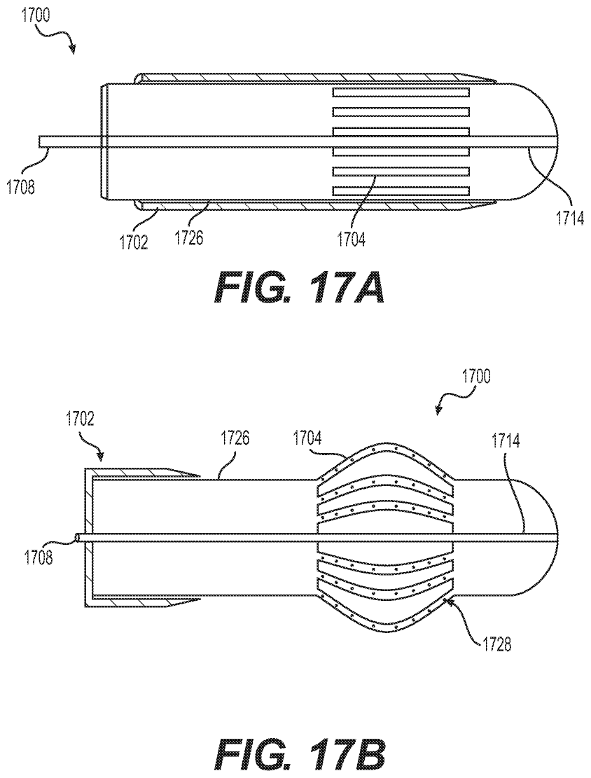

FIG. 17A depicts an interventional device 1700 with a delivery sheath 1702 covering a cutting element 1704, which is cut from a polymer tube 1726 and, as such, is integral to the tube 1726. In an alternative embodiment (not shown) of the design shown in FIG. 17A, the device 1700 includes an additional element that would be positioned in a lumen after removal of a guidewire. This element may serve to deliver fluid for flushing. In this example, aspiration could be accomplished by applying suction to a proximal end of the sheath 1702, such that fluid is removed as well as debris while flushing is activated.

FIG. 17B shows a moveable internal corewire 1708 that extends down an inner lumen of the polymer tube 1726 and is fastened to the distal tip 1714 of the device 1700 to provide a means for deploying the cutting element 1704 and a distal protection element through either expansion or contraction. Abrasives 1728, which are mounted, coated, or integral with the cutting element 1704, may be designed to facilitate material removal and shaping of the lesion.

### Other Embodiments of Interventional Device

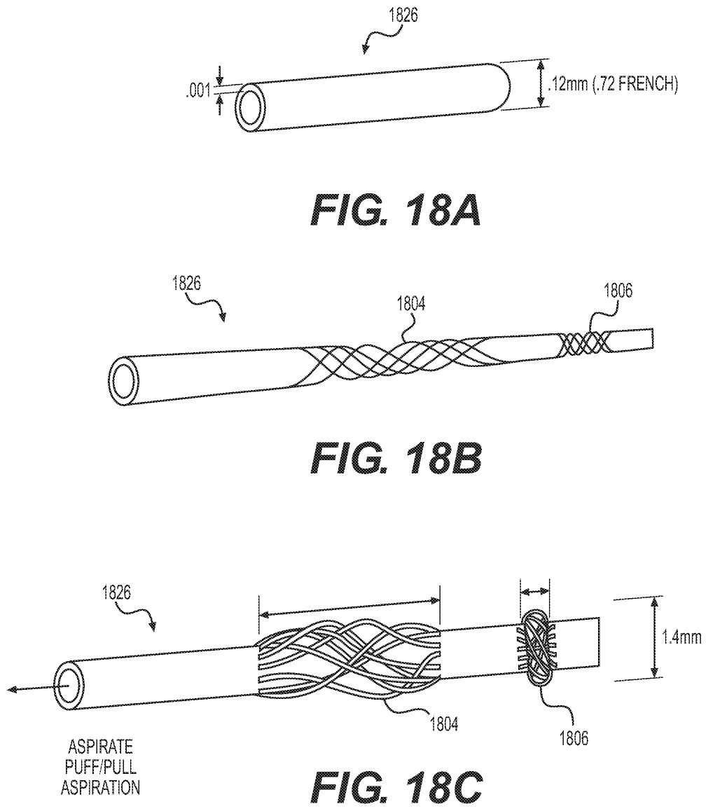

FIG. 18 shows a series of schematic drawings of a single hypotube-based design in which a “puff/pull” aspiration of atherectomy debris is applied. A single hypotube 1826 of 0.12 (0.10-0.14) mm with a 0.001″ thickness is laser cut and set to expand an interventional device, such as an atherectomy device 1804, and a distal protection device 1806 thereof. The device may be used by puffing saline or another inert liquid into the space while simultaneously (manually or mechanically) aspirating a diseased area and applying a rotational force (pushing or turning, mechanically or manually) on the lesion. When fully deployed, the device is 1.4 mm in maximum diameter.

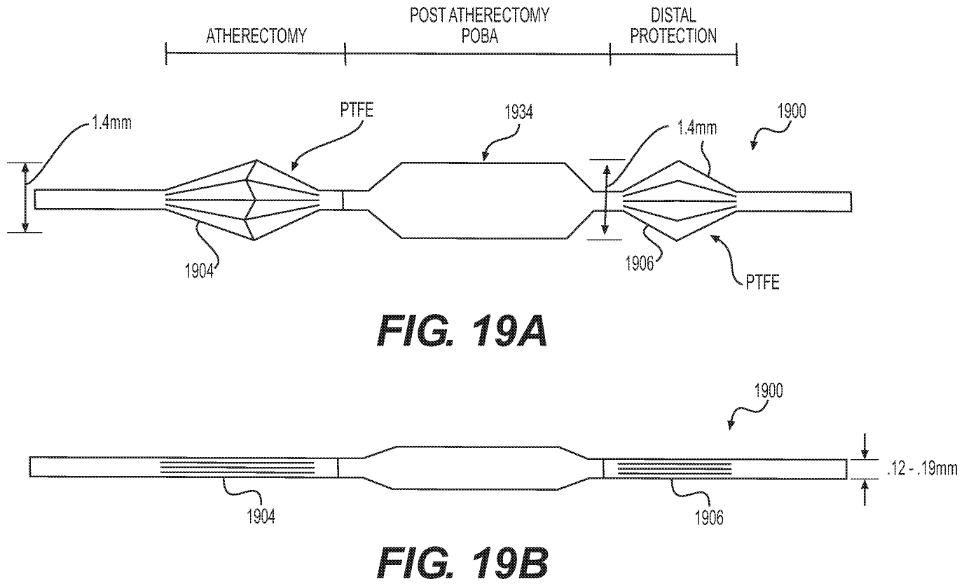

FIG. 19 shows a series of schematic drawings of a basket-like interventional device 1900, such as an atherectomy device 1904, proximal to a POBA/DE balloon 1934, proximal to a distal protection device 1906. The basket-like atherectomy device 1904 and distal protection device 1906 are deployed distally to a lesion. The basket-like atherectomy device 1904 is pulled into a catheter (not shown), scraping debris into a basket of the basket-like interventional device 1904. As the balloon 1934 passes the lesion site after an atherectomy, an angioplasty is applied, facilitating a smooth, non-striated blood interface.

### Balloon-Based Interventional Device

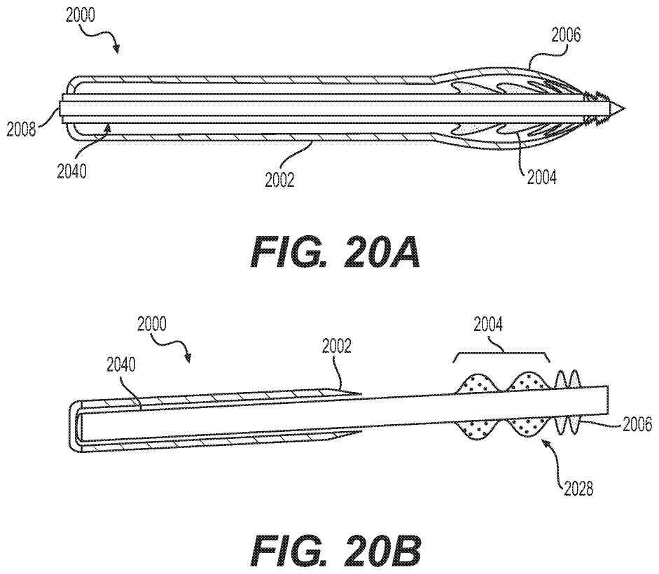

FIG. 20A depicts the device 2000 with the delivery sheath 2002 covering a balloon catheter body 2040 as well as the cutting element 2004 and the distal protection element 2006, which are both integral with the balloon catheter body 2040. (Note: There would also be a provision for an element that would be positioned in the lumen after removal of a guidewire. This element may serve to deliver fluid for flushing. In this example, aspiration could be accomplished by applying suction to the delivery sheath 2002, such that fluid is removed as well as debris while flushing is activated. While this arrangement is not sketched, this document discloses such configuration). The guidewire 2008 may extends down (e.g., along, through) an inner lumen of the device 2000 to provide a means for navigating the anatomy.

FIG. 20B depicts placement of the device 2000 within the target anatomy, where the guidewire 2008 is removed and the delivery sheath 2002 is pulled back, exposing the cutting element 2004 and the distal protection element 2006. Deployment of the distal protection element 2006 may be controlled by use of an inflation device to fill a balloon, which may be the distal protection element 2006, with fluid. Once the balloon is inflated, the profile may take shape such that the cutting element 2004 and the distal protection element 2006 are deployed. Abrasives 2028 are mounted, coated, or integral with the cutting element 2004, and may be designed to facilitate material removal and shaping of a lesion. Hydrogels or another material may be integral to the distal protection element 2006 such that a material is attracted and adheres to it.

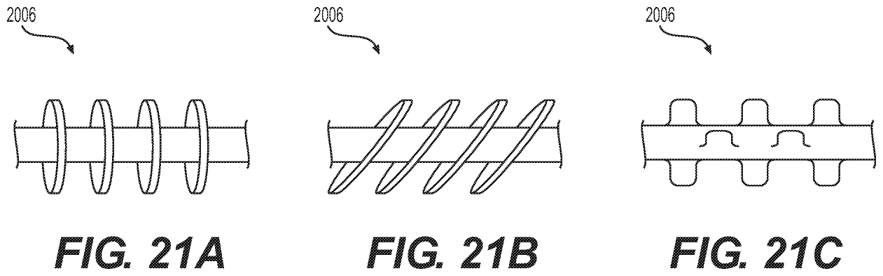

FIGS. 21A-21C depict some general shapes for the balloon as the distal protection element 2006. These sketches serve to provide only general variation ideas, and are not meant to be all inclusive.

### Interventional Device—Ophthalmic Artery Access Element

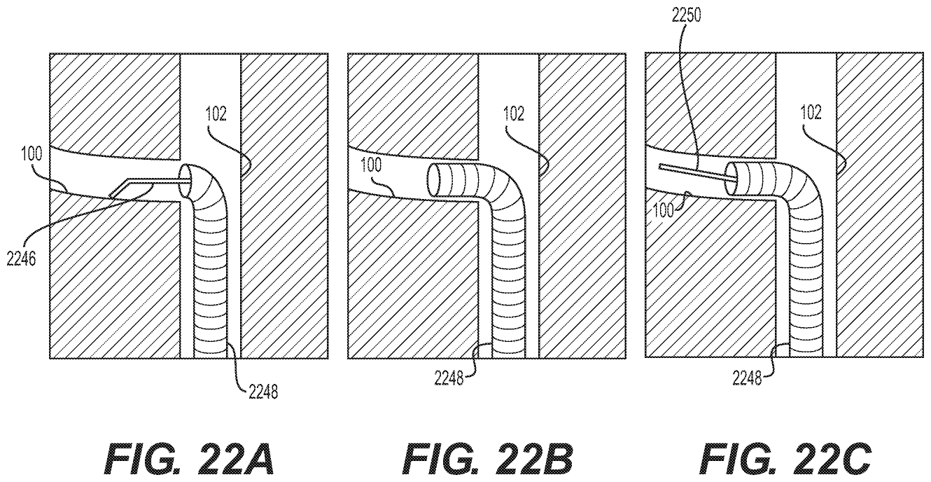

FIGS. 22A-22C show the use of a shaped guidewire 2246 to access the OA and follow up with a guiding catheter 2148 to position within the entry to the OA. Once inside, the shaped guidewire 2146 may be exchanged for either a straight guidewire 2150 or an interventional device to continue the procedure. FIGS. 22A-22C depict access of the OA by use of a shaped guidewire 2246, entry into the OA by a guiding catheter 2248 over the shaped guidewire 2246, and finally exchange of the shaped guidewire 2246 for either a straight tip guidewire 2250 or an interventional device. The guidewire 2246 and the guiding catheter 2148 are specifically designed for use in the OA, and may include a provision for providing downstream protection.

### Complete Temporary Arterial Evacuation

In one embodiment, once the retrograde flow is established, a shunt tube is used to continuously drain the target artery, while the atherectomy device is used to remove the plaque. Since the target artery is being subjected to pressure from an intraocular pressure measuring device (IOP means) at a distal (antegrade) location, the entire vessel contents, including the plaque particles, may be bled off, thus reducing the risk of plaque-associated ischemic damage. Compared to the aortic flow rate of up to 5 L/min to 6 L/min, or slightly less for the coronary arteries, the technique of using complete temporary arterial evacuation is possible precisely because the ophthalmic, retinal, and other eye vasculature is significantly smaller, and blood loss can be kept to acceptable levels during an atherectomy of an ophthalmic artery.

### Distal Protection, Mesh Basket, and the Interventional Device

FIGS. 19A and 19B show an example of an interventional element or interventional device 1900. The dimensions noted are illustrative and non-limiting. However, for use in the OA, ST, and SO, the dimensional requirements, and, in particular, the remarkably narrow diameter, allows for access to and treatment of narrow and peripheral arteries and blood vessels, such as are in the eye. To date, interventional atherectomy of the ophthalmic artery (OA), and related vessels, has not been disclosed as described in the claims herein.

As noted above, FIGS. 19A and 19B show a basket-like atherectomy device 1904, proximal to a POBA/DE Balloon 1934, proximal to a distal protection device 1906. The basket like atherectomy device 1904 and distal protection device 1906 are deployed distal to the lesion. The device 1900 is pulled into a catheter, scrapping debris into the basket 1904. As the balloon 1934 passes the lesion site after atherectomy, an angioplasty is applied, facilitating a smooth, non-striated blood interface. Various atherectomy devices are disclosed in U.S. Pat. No. 6,010,522, which is incorporated herein by reference in its entirety.

Other exemplary filter devices are available from Boston Scientific, Medtronic Vascular, and so forth, such as those disclosed in U.S. Pat. Nos. 8,409,237, 8,267,956, 8,123,779, 6,974,469, and others disclosed in Class 606 surgical devices, with subclass 200 emboli traps or filters, all incorporated by reference herein in their entireties.

The references recited herein are incorporated herein in their entireties, particularly as they relate to teaching the level of ordinary skill in this art and for any disclosure necessary for the commoner understanding of the subject matter of the present disclosure. It will be clear to a person of ordinary skill in the art that the above embodiments may be altered or that insubstantial changes may be made without departing from the scope of the present disclosure. Accordingly, the scope of the invention is determined by the scope of the following claims and their equitable equivalents.

What is claimed is:

1. A method, comprising:
accessing a terminal branch of an ophthalmic artery through a skin of a head of a subject via a first device;
positioning at least one of the first device or a second device within the ophthalmic artery of the subject; and
performing an atherectomy of the ophthalmic artery or a junction between the ophthalmic artery and an internal carotid artery of the subject, using the at least one of the first device or the second device, so as to treat a blockage, a stenosis, a lesion, plaque, or other physiology in the ophthalmic artery or the junction between the ophthalmic artery and the internal carotid artery of the subject.

2. The method of claim 1, wherein the performing the atherectomy includes increasing a size of a lumen of the ophthalmic artery or the junction between the ophthalmic artery and the internal carotid artery of the subject.

3. The method of claim 1, wherein the accessing the terminal branch of the ophthalmic artery through the skin of the head of the subject includes accessing one of a supraorbital artery, a dorsal nasal artery, or a supratrochlear artery of the subject via the first device.

4. The method of claim 1, further comprising measuring a blood flow rate in the ophthalmic artery.

5. The method of claim 1, further comprising inhibiting antegrade passage of debris via a distal protection element.

6. The method of claim 1, further comprising performing an angioplasty of the ophthalmic artery or the junction between the ophthalmic artery and the internal carotid artery of the subject.

7. The method of claim 1, wherein the accessing the terminal branch of the ophthalmic artery through the skin of the head of the subject via the first device includes accessing the terminal branch via a cut-down procedure.

8. The method of claim 1, wherein the performing the atherectomy includes performing the atherectomy via an atherectomy element including a basket-shaped structure.

9. A method, comprising:
accessing a terminal branch of an ophthalmic artery through a skin of a head of a subject via a first device;
positioning at least a portion of the first device or a second device within the ophthalmic artery distal of an area to be treated within the ophthalmic artery, wherein the first device includes an atherectomy element; and
treating tissue of the ophthalmic artery by withdrawing the at least the portion of the at least one of the first device or the second device toward the area to be treated to increase vascular flow through the ophthalmic artery.

10. The method of claim 9, wherein the withdrawing the portion of the at least one of the first device or the second device causes tissue debris.

11. The method of claim 10, further comprising capturing the tissue debris and removing the tissue debris from the subject.

12. The method of claim 9, further comprising performing an angioplasty on the area to be treated.

13. The method of claim 9, wherein the accessing the terminal branch of the ophthalmic artery through the skin of the head of the subject includes accessing one of a supraorbital artery, a dorsal nasal artery, or a supratrochlear artery of the subject via the first device.

14. The method of claim 9, further comprising measuring a blood flow rate in the ophthalmic artery.

15. The method of claim 9, wherein the treating the tissue of the ophthalmic artery includes treating an eye disease, wherein treating the eye disease comprises treating macular degeneration.

16. A method, comprising:
positioning a first device in a terminal branch of an ophthalmic artery through a skin of a head of a subject, wherein positioning the first device in the terminal branch of the ophthalmic artery through the skin includes positioning the first device in one of a supraorbital artery, dorsal nasal, or a supratrochlear artery;
positioning at least a portion of the first device or a second device within the ophthalmic artery distal of an area to be treated within the ophthalmic artery, wherein the at least the portion of the first device or the second device includes an atherectomy element; and
performing an atherectomy on the area to be treated by moving the atherectomy element relative to the area to increase vascular flow through the ophthalmic artery.

17. The method of claim 16, further comprising performing an angioplasty on the area.

18. The method of claim 16, wherein the moving the atherectomy element relative to the area to be treated causes tissue debris.

19. The method of claim 18, further comprising capturing the tissue debris and removing the tissue debris from the subject.

20. The method of claim 16, further comprising measuring a blood flow rate in the ophthalmic artery.
